(19) United States
(12) Patent Application Publication
(10) Pub. No.: US 2026/0199104 A1
(43) Pub. Date: Jul. 16, 2026

(54) ORTHOPAEDIC DEVICE AND METHOD FOR CONTROLLING SAME

(71) Applicant: OTTO BOCK HEALTHCARE PRODUCTS GMBH, Wien (AT)

(72) Inventor: Dirk SEIFERT, Wien (AT)

(21) Appl. No.: 19/137,618

(22) PCT Filed: Dec. 13, 2023
(86) PCT No.: PCT/EP2023/085624

§ 371 Date: Aug. 28, 2025

(30) Foreign Application Priority Data
Dec. 15, 2022 (DE) 10 2022 133 462.7

(51) Int. Cl. A61F 2/70; A61F 2/64; A61F 2/66; A61H 3/00

(52) U.S. Cl. CPC A61F 2/70; A61F 2/64; A61F 2/6607; A61H 3/00; A61H 2201/5069; A61H 2201/5084

(57) ABSTRACT

The invention relates to a method for controlling an orthopaedic device (100) for the lower extremity, having a proximal top part (2) and a distal bottom part (3), which are hinged to one another about at least one pivot axis (4) so as to form a joint (5), having devices for attaching the orthopaedic device to a limb, and at least one actuator (6) which is coupled to a control device (7) which activates or deactivates the actuator (6) on the basis of sensor data from at least one sensor (8) coupled to the control device (7), in order to influence a pivoting resistance and/or a movement of the top part (2) relative to the bottom part (3), or of two components of the orthopaedic device relative to one another, wherein an orientation and/or change in orientation about the longitudinal axis of the longitudinal extent in the proximal-distal direction of the orthopaedic device (100) and/or of a contralateral limb is/are detected using the sensor data, and the actuator (6) is activated or deactivated or a target value for the actuator (6) is modulated on the basis of the orientation and/or change in orientation about the longitudinal axis.

### CROSS REFERENCE TO RELATED APPLICATIONS

The present application is the U.S. National Phase under 35 U.S.C. § 371 of International Application No. PCT/EP2023/085624, filed on 13 Dec. 2023, which claims priority from German Patent Application No. 10 2022 133 462.7, filed on 15 Dec. 2022, the contents of which are hereby incorporated by reference in their entirety.

The invention relates to a method for controlling a lower-limb orthopedic device having an upper part and a lower part, which are mounted on each other in an articulated manner about at least one pivot axis so as to form a joint with each other, and having at least one actuator coupled to a control device which activates or deactivates the actuator on the basis of sensor data from at least one sensor coupled to the control device, in order to influence a pivoting resistance or a movement of the upper part relative to the lower part, and it also relates to such an orthopedic device, in particular for carrying out the method.

Orthopedic devices of the lower limbs are understood in particular to be orthoses and prostheses. Orthoses are orthopedic aids which are applied to an existing limb and which guide, restrict or support movements. Drives, actuators and/or resistance devices, which can be adjusted or set via an actuator, can be arranged between components that are connected to one another in an articulated manner. The adjustment can be effected on the basis of sensor data that are transmitted to a data processing device. In the context of this application, orthoses are also understood to mean exoskeletons that are attached to the body of a patient and form an external support structure, in particular to guide and influence the movements of a user, e.g. to support them by drives or to brake them via resistance devices. Orthoses, and exoskeletons as special cases thereof, can be used and employed not only for assistance in everyday activities, but also for training purposes or for therapeutic purposes.

Prostheses replace limbs that are not present or no longer present. The simplest prosthesis components have a purely cosmetic function or complete a limb, for example by replacing a distal phalanx. Over the course of time, prostheses have become more complex, multiple prosthesis components have been arranged and fastened on one another and, for example, connected to one another via joints. Complex mechanical drive devices have been developed to move prosthetic hands or prosthetic feet, for example. Hydraulic or other damping devices or resistance devices have been arranged on joints in order to modify the behavior of prosthetic components and prosthesis systems in order to enable the most natural movement sequence possible. To support movements, drives have been integrated into prosthesis components in order to create active prostheses. Furthermore, sensors have been arranged on prosthetic components or on a person using the prosthesis, in order to record the current movement behavior or the current positions of prosthetic components in relation to one another and to estimate future movement behavior and to change settings on resistance devices and/or drives. This has resulted in highly complex prosthetic systems with multiple prosthetic components arranged on one another, which have a large number of mechanical, electrical and mechatronic components.

A prosthetic system of the lower limb can in particular have a thigh socket, to the distal end of which a prosthetic knee joint, a prosthetic lower leg and a prosthetic foot are attached. Such a prosthetic system has, for example, two or more joints, each of which can be provided with resistance devices and/or drives or actuators.

EP 2 816 979 B1 discloses a method for controlling an artificial orthotic knee joint or prosthetic knee joint, in which method the flexion resistance is changed based on the detection of an absolute angle of a lower-leg component. The determined absolute angle of the lower-leg component is compared against a threshold value; if the threshold value is reached or exceeded, the flexion resistance is changed.

EP 2 649 968 B1 discloses a method for controlling an orthopedic foot part having an ankle joint, in which method the torques occurring at the ankle joint, the ankle angle and the absolute angle of a foot part in relation to the vertical are determined.

Depending on the measured values, the rolling of the foot in the stance phase, the position of the foot part in the swing phase and the position and mobility of the foot part while standing are controlled by means of a damping arrangement.

In order to switch between different operating modes, it is also known for orthopedic devices for the lower limbs to be subjected to particular loading in order to set a special mode. Repeated, rhythmic loading within a certain period of time and in a certain direction of loading is considered as a switching signal in order to then activate special programs, for example for walking up stairs. Conscious switching of the operating modes requires a high level of attentiveness on the part of the person using the orthopedic device.

The object of the present invention is to make available a method and an orthopedic device with which users of orthopedic devices of the lower limbs can more easily carry out activities of everyday life and with which it is possible to achieve a more versatile use of the orthopedic device.

According to the invention, this object is achieved by a method having the features of the main claim and by an orthopedic device having the features of the additional independent claim. Advantageous embodiments and developments of the invention are disclosed in the dependent claims, the description and the figures.

In the method for controlling a lower-limb orthopedic device having a proximal upper part and a distal lower part, which are mounted on each other in an articulated manner about at least one pivot axis so as to form a joint with each other, means for fastening the orthopedic device to a limb, and at least one actuator coupled to a control device which activates or deactivates the actuator on the basis of sensor data from at least one sensor coupled to the control device, in order to influence a pivoting resistance and/or a movement of the upper part relative to the lower part or of two components of the orthopedic device relative to each other, provision is made that an orientation and/or change in orientation about the longitudinal axis of the longitudinal extent in the proximal-distal direction of the orthopedic device and/or of a contralateral limb are detected using the sensor data, and the actuator is activated or deactivated, or a setpoint value for the actuator is modulated, on the basis of the orientation and/or change in orientation about the longitudinal axis. Whereas in the prior art the movements in the sagittal plane are evaluated and used to modify resistances or drives, according to the invention a displacement about a longitudinal axis of the longitudinal extent of the orthopedic device or of the contralateral limb or about both longitudinal axes is recorded and used to influence the resistances or drives of the orthopedic device accordingly. Displacements about the longitudinal axis occur particularly when turning, for example when walking around a bend, making abrupt changes in direction, participating in sporting activities and the like. By taking such movements into account, it is possible, using microprocessor control, to adapt the orthopedic device to the respective movements and situations that deviate from straight-line movements in the sagittal plane, particularly in the swing phase, e.g. when walking around a bend. Such control processes are also advantageous and useful in orthopedic devices for the upper limbs, in order to be able to respond to the large number of possible movements and movement patterns that occur when performing everyday activities and to avoid having to accept unnecessary restrictions.

In a further development, provision is made that the sensor data are determined and the actuator is activated or deactivated, or the setpoint value for the respective actuator is modulated, during use of the orthopedic device in the fitted state. This makes it possible to influence the pivoting resistance and/or a movement of the upper part relative to the lower part, adapted to the respective movement and to the respective movement situation.

In one embodiment, the orientation or displacement of the orthopedic device or of parts of the orthopedic device is detected and determined using a spatial position sensor, at least one IMU (inertial measurement unit) and/or at least one angle sensor. The angle sensors or the angle sensor detect(s) the position of the upper part relative to the lower part, of individual components to one another or of the components relative to a body part or another reference element on the user and make it possible to determine the orientation of the entire orthopedic device, several parts thereof or just one part thereof, with regard to the respective position, orientation and movement about a longitudinal axis along the longitudinal extent of either the orthopedic device or the contralateral limb or the torso. The orientations of components or of the entire orthopedic device and, if necessary, the orientation of the components relative to one another can be determined directly using a spatial position sensor or an IMU. An IMU can be designed in such a way that a magnetic field, in particular the earth's magnetic field, is detected in one direction or several directions and used to calculate the orientation. This makes it possible to determine the orientation of a component or of several components with respect to the magnetic field. After the evaluation, corresponding commands are transmitted to the control to activate, deactivate or modulate a setpoint value of the actuator in order to change the pivoting resistance and/or the relative movement. A relative angle between two components can be determined from the respective absolute angles, for example using two IMUs. From the absolute angle or solid angle of a component and from the relative angles with respect to other components, it is possible to determine the absolute angle thereof.

In a further development, forces, moments and/or accelerations of the entire orthopedic device and/or of its components are recorded via sensors and also used as a basis for the control. In connection with the recording of pivoting movements and/or displacements, e.g. within the frontal plane and/or in the sagittal plane, additional parameters are used to control the actuator. The absence of axial forces shows, for example, that the orthopedic device is in a swing phase. Different force distributions or introduction of moments about pivot axes enable movements, changes in movement, states and likely future movements or loads to be determined, so that the actuator is supplied with appropriate commands based on the forces, moments and/or accelerations, in particular in conjunction with data on the orientation and/or displacement about the longitudinal axis of the longitudinal extent in the proximal-distal direction. Forces and moments can be determined via a deformation, displacement, tilt and/or a combination of these. For example, an acting force and/or a moment can be inferred from the deformation of an elastic or compressible body and/or from a resulting tilt or displacement. A force and/or a moment can also be inferred from the displacement or deformation rate of a damper or viscous element.

In a further development, at least one orientation and/or change in orientation about the longitudinal axis of the orthopedic device and/or of a contralateral limb and/or of the body is estimated or calculated on the basis of sensor data and a model. Models can be used to calculate or estimate quantities that are not accessible or only with difficulty accessible by measurement. Mechanical models can be used to calculate movements from forces and moments using equations of motion. State variables can be continuously estimated from other sensor data using filters, for example Kalman filters. State variables such as orientation and/or change in orientation can be estimated from other sensor data using artificial intelligence algorithms such as neural networks, recursive neural networks, convolutional neural networks, state vector machines, linear discriminant analysis, K-means or regression. The estimation and/or calculation can be carried out continuously or at specific times.

In a further development, the orientation and/or change in orientation about the longitudinal axis of the orthopedic device and/or a contralateral limb and/or the body of at least one component is determined by means of an environmental sensor system, in particular by detecting electromagnetic radiation, in relation to a reference. The reference can be the environment and/or a contralateral limb. The electromagnetic radiation can be emitted by the orthopedic aid for the measurement. The environment can be recorded and the orientation and/or change in orientation can be determined using one or more cameras, depth imaging cameras, radar and/or lidar sensors and the like. The distance to one or more objects in the environment, and thus the orientation and/or change in orientation, can be determined using time-of-flight measurements of electromagnetic radiation, but also using sonar. The orientation and/or change in orientation can be determined using global navigation systems and/or indoor navigation, for example using beacons or WLAN. It is also possible for the orientation and/or change in orientation of the orthopedic aid to be recorded via an external system, for example via one or more cameras, depth imaging cameras, radar and/or lidar sensors, and for the information to be transmitted to the aid. The orientation and/or change in orientation can be determined using positions that follow one another in time and that are determined using the environmental sensor system.

In one embodiment, the orthopedic device is designed as a prosthesis or orthosis and has an artificial knee joint and/or ankle joint, with an actuator being assigned to each joint. If both a knee joint and an ankle joint are present, two actuators can be present in order to individually and independently change a pivoting resistance about the respective joint or to initiate or influence a relative movement between the respective upper part and the respective lower part. It is also possible for just a single actuator to be assigned to two joints, via which actuator a corresponding increase or reduction in a pivoting resistance is achieved or a shift from upper part to lower part is effected. It is also possible for more than two joints and one or more actuators to be arranged and controlled.

In a further development, the at least one sensor is arranged on the orthopedic device, the contralateral limb or the torso of the user, or several sensors are arranged on the orthopedic device, the contralateral limb and/or the torso of the user. Sensor data are determined via this sensor or sensors, which sensor data then form the basis for further control of the actuator. By the arrangement of sensors, it is possible not only to record and control the absolute rotation of the treated side about the longitudinal axis, but also or alternatively to take into account the rotation relative to the body. In particular, the relative movement and position of the treated side with respect to the rest of the body is preferably recorded using several sensors, since the relative displacements and relative rotations can easily be derived from the various sensor values. The IMUs in particular permit a comparatively precise, simple and inexpensive determination of movements and positions in space in different planes and about different axes, so that the existing movement can be easily deduced by evaluating their data.

In one embodiment, it is provided that, during a rotation of the treated side about the longitudinal axis of the longitudinal extent of the contralateral limb in the swing phase of the orthopedic device, an increase in flexion resistance is initiated, in particular compared to when walking straight ahead. Alternatively or in addition, a reduction in extension resistance is initiated, or extension is actively initiated or supported, or an already existing support is increased. In contrast to pure movements in the sagittal plane, the movements during rotations about the longitudinal axis of the longitudinal extent of the contralateral limb, in particular of a leg, can take significantly longer, so that the usual control mechanisms cannot be used or cannot be used as effectively. When walking around a bend, it can happen that the knee joint does not extend in good time before set-down, because the time periods differ from when walking on the level. This occurs primarily when walking around a bend, when an internal rotation around the contralateral supporting leg is carried out in the swing phase of the treated side. Such a movement is carried out, for example, when changing direction. In the presence of such a rotation, it is advantageous to increase the flexion resistance in the swing phase of a passive knee joint, in order to reduce the maximum knee flexion angle. This leads to a shortened pendulum movement compared to when walking straight ahead on the level. Energy that is additionally introduced into the system by the user during the rotation, compared to when walking straight ahead, can be dissipated by increased resistance and/or by resistance that acts for a longer period. An extension resistance can be reduced as compared to when walking straight ahead, in order to ensure extension as quickly as possible. If an active knee joint is provided with motor support or another release of a stored amount of energy, the extension can be actively supported and, in particular, can be supported more strongly and/or earlier than when walking straight ahead. This makes it possible to control both the flexion movement and the extension movement so that they correspond with the rotational movement around the supporting leg. The extension can thus be initiated when a reduction of the rotational movement or a slowing-down of the rotational movement is detected.

In a further development of the method, provision is made that, during a rotation of the treated side about the longitudinal axis of the longitudinal extent of the contralateral limb in the swing phase of the orthopedic device, hip flexion is supported, for example via a motor or by release of an energy storage device such as a spring or a pneumatic accumulator. Alternatively or in addition, hip flexion resistance can be reduced so that hip flexion can be initiated more strongly or more quickly, as a result of which the resistances or support rates are adapted to the respective rotational movement.

In a further development, during a rotation of the treated side about the longitudinal axis of the longitudinal extent of the treated side in the stance phase, the flexion resistance is increased, an ongoing or existing flexion is reduced, and/or a flexion or further flexion is prevented. In particular, a resistance of an ankle joint in dorsal flexion can be increased compared to when walking straight ahead, possibly to the point of locking; alternatively, plantar flexion can also be supported. The flexion resistance of the knee joint can also be increased as compared to when walking straight ahead, a flexion movement can be stopped and/or an extension movement in the knee can be supported. Both the time profiles and the level of the resistance and/or support moments can be adjusted. With such an increase of the resistance or an extension or locking of further flexion, it is possible to facilitate the execution of the rotational movement during rotation of the treated side as the supporting leg and to prevent unwanted flexion. This increases stability and safety for the person using the orthopedic device. Such an increase in flexion resistance or extension can take place, for example, in the ankle joint, knee joint and/or hip joint.

The swing phase in a rotational movement can last longer than when walking straight ahead. This is especially the case when it is a rotation through a large angle, for example when turning around with a rotation of 180°, but also in the case of complete or multiple rotations about the body axis, which can occur in special situations. In this case, it is advantageous to control the orthopedic device in such a way that sufficient ground clearance is achieved during the entire swing phase. With an artificial leg, this can be achieved by knee flexion, dorsal extension in the ankle joint, and/or possibly hip flexion. With a control that is typical for walking straight ahead, the knee joint for example would extend too early in such situations after bending and the foot would catch on the ground. In a further development, therefore, a rotation, in particular a more protracted rotation, is detected and the control of the hip, knee and/or foot is then adjusted; in particular, a hip flexion, a knee flexion and/or a dorsal flexion in the foot after a swing phase flexion initiation is supported and/or maintained for longer than when walking straight ahead. Alternatively or in addition, the movements of one or more joints are slowed down in order to adapt the movement sequence to the longer-lasting rotation and to prevent extension taking place too early. If a slowing down and/or an end of the rotational movement is detected, the resistances and/or the drives in the hip, knee and/or foot are controlled in such a way that the knee joint is extended and the foot is brought into a position that is advantageous for the initial contact, for example by initiation of an extension movement or reduction of an extension resistance.

In a further development, in the event of a change in orientation about the longitudinal axis of the longitudinal extent of the treated side, the contralateral limb and/or the body, a flexion resistance in the stance phase of the treated side, especially in the terminal stance phase, is not reduced and/or a flexion is not supported. Alternatively or in addition, a smaller reduction in the flexion resistance and/or less support of the flexion movement is carried out than when walking straight ahead. When walking straight ahead, flexion resistance in an artificial knee joint is typically reduced in the terminal stance phase or flexion is released in order to enable slight bending in the swing phase initiation. In an active knee joint, the flexion movement is actively supported in order to achieve particularly slight bending and a sufficiently high knee flexion angle. In an active ankle joint, a plantar flexion moment is generated in the terminal stance phase in order to push the foot and thus also the body forward in the direction of walking. In the case of a rotation with the treated side as the supporting leg, it can be advantageous not to reduce the flexion resistance in the knee joint, or to reduce it only to a lesser extent, in order to prevent unwanted or unexpected bending. In such an embodiment, the flexion resistance is left at a typical stance phase level or only partially reduced when a rotation is detected. In an active knee joint, no flexion movement is initiated or supported, or the support of the flexion movement is reduced. In an active foot, active plantar flexion is not initiated or is supported less than when walking straight ahead. This can be an advantage over existing systems, particularly in the case of people with an increased need for stability or with reduced coordination skills. Particularly when changing direction and turning within a confined space, an unexpected reduction in flexion resistance or an unexpected initiation of a flexion movement can lead to instability, loss of balance or a fall.

In one embodiment, a special mode is exited during a rotation about the longitudinal axis of the orthopedic device and/or a contralateral limb and/or the body.

Alternatively or in addition, switching to a special mode is prevented or aborted during such a rotation. Orthopedic devices often have several operating modes. In addition to a basic mode, there are one or more special modes for special movement sequences or activities. Special modes can cover cyclical movement sequences that deviate from walking on the level, such as climbing stairs, walking uphill or running. Special modes can also cover quasi-static situations, such as sitting or standing bent over. Last but not least, special modes can cover special cyclical or non-cyclical activities, such as cycling, playing table tennis, skiing or a standby mode. In a special mode, the resistances and actuators of the orthopedic devices are controlled differently than in the basic mode or when walking on level ground. For example, when climbing stairs, the relief of the treated side supports an active flexion movement with a high range of motion in the knee joint for placing the foot upwards, and an extension movement in the subsequent stance phase for lifting the body. In a bicycle mode, the resistances in the knee joint are reduced to a minimum, or an extension movement when pedaling downward is actively supported. Switching to a special mode can be done autonomously by the orthopedic device on the basis of sensor values. For example, based on the trajectory of the foot recorded in the sagittal plane in a swing phase, it can be concluded that the activity involves climbing stairs, and a switch can be made to a stair-climbing mode. The cyclical pedaling movement when riding a bike can also be detected and a switch can be made to a cycling mode. Switching back to a basic mode can also be done autonomously on the basis of sensor values. Alternatively or in addition, movement patterns such as rocking on the forefoot several times, control elements and/or external devices such as smartphones or tablets, which communicate with the orthopedic device, can be used for switching to a special mode and back again. The combination of autonomous and non-autonomous switching is also possible. The control in a special mode is advantageous for a specific movement and/or a specific activity. In other movements and/or situations, this control can be disadvantageous or unsafe. For many movement sequences and situations, a rotation about the longitudinal axis, especially a rapid rotation or a rotation with a large range of motion, is atypical and can serve as an indicator that the original movement sequence should be switched to another one and that the control or the underlying control law should be adjusted. The rotation about the longitudinal axis can therefore serve to detect a change in the situation and/or the movement mode and then to exit a special mode. For example, when climbing stairs, a rotation about the longitudinal axis of the contralateral side can be detected and the stair-climbing mode can then be exited or not activated. This can prevent accidental knee flexion in such a situation in the swing phase of the treated side, especially when the user turns around and wants to go down the stairs. If a bending movement in the knee joint has already been initiated before the rotation is detected, the knee joint can be extended due to the rotation when leaving the special mode for climbing stairs, in order to enable the load to be transferred to the treated side.

In a further development, in order to provide adapted control even in special situations such as skiing, an extension lock is canceled, especially when it is detected that the downhill ski is becoming an uphill ski. This is done, for example, by detecting a rotation about the longitudinal axis of the treated and/or contralateral limb with a bent knee and/or a relief of the axial forces acting on a lower leg. If a swing is detected, an extension lock of the downhill ski can be canceled, for example if a rotation threshold value is detected.

In one embodiment of the method, the orientation or the change in orientation about the longitudinal axis of the longitudinal extent in the proximal-distal direction of the orthopedic device and/or of a contralateral limb is recorded relative to the torso of the person using the orthopedic device, to a stationary component of the orthopedic device and/or to an external reference orientation, for example relative to the gravity orientation. Thus, for example, if an ankle joint can be rotated about the longitudinal axis in the context of an internal rotation or external rotation, then a rotation of the thigh about the longitudinal axis can be increased, this being possible both in the swing phase and in the stance phase. A torsion adapter can be arranged between a prosthetic knee joint and a thigh part or thigh socket, via which torsion adapter, in the case of a passive prosthetic foot with an adjustable rotation resistance about the longitudinal axis, it is possible to increase the possible range of motion in the form of the pivot angle when a rotational movement is detected in the stance phase, in order to increase mobility. This can facilitate the corresponding rotation or make it possible in the first place. A change in the rotation resistance is particularly useful when a total rotation of the orthopaedic device is detected, i.e. when turning on the foot of the treated side is detected, in order then to achieve increased flexibility within the orthopedic device.

In one embodiment, the actuator is activated or deactivated, or a setpoint value for the actuator is modulated, depending on the duration, extent, speed and/or speed profile of the change in orientation and/or of a movement. In particular, the rotation about a longitudinal extent in the proximal-distal direction is taken into account, if necessary in conjunction with other movements or measured variables that are recorded via the sensors. Preferably, the change in the resistances or the activation of the actuator or the modulation is carried out in combination with a large number of other sensor values in order to achieve greater control accuracy. For example, the maximum swing phase flexion angle can be changed and the level of extension resistance in the swing phase can be changed. When the orthopedic device is actively adjusted via a drive, it is possible to use moments, angles, positions, inoculations, stiffnesses, speeds, admittances or impedances as control variable.

In one embodiment, the sensor is designed as an IMU and is attached to the treated limb, the contralateral limb and/or the torso of the patient. A relative rotation of the limb or limbs with respect to the torso is detected via the IMU, and, on the basis of the detected rotation, the resistance is changed accordingly or a movement is supported or initiated.

A further development provides that a trajectory of the orthopedic device and/or of the contralateral limb is recorded and used as the basis for the activation or deactivation of the actuator or the modulation of a setpoint value of the actuator. In particular, the trajectories are determined via one or more IMUs or an environmental sensor system in order to detect a rotation about a corresponding longitudinal axis. In the case of a rotational movement as well as a change of direction, the trajectory of the respective component or limb, in particular in the transverse plane, also has a curved shape, so that the trajectory and its features, such as topology, shape, length, tangent orientation and/or their temporal changes and/or curvature, can be used to infer an existing rotation.

In the lower-limb orthopedic device having an upper part and a lower part, which are mounted on each other in an articulated manner about at least one pivot axis so as to form a joint with each other, and at least one actuator coupled to a control device which activates or deactivates the actuator on the basis of sensor data from at least one sensor coupled to the control device, in order to influence a pivoting resistance or a movement of the upper part relative to the lower part, provision is made that the at least one sensor is designed and configured to detect sensor data concerning an orientation and/or change in orientation of the orthopedic device about the longitudinal axis of the longitudinal extent in the proximal-distal direction of the orthopedic device and/or of a contralateral limb, and that the control device is configured to activate or deactivate the actuator, or modify a setpoint value for the actuator, on the basis of the orientation and/or change in orientation about the longitudinal axis. The actuator is used to move the upper part relative to the lower part, to block a movement between the upper part and the lower part, to provide resistance to such a movement or to modulate such a movement. This is done, for example, by introducing energy into the system. The change in resistance or the drive is effected in accordance with the detected rotational movement, wherein the control not only affects repeated gait cycles, but in particular also movements in which the orthopedic device is repositioned under the body without loading. An example of this is movement of the leg from a bent position in combination with a rotation about the longitudinal axis, for example from a standing position or a seated position. In such a movement, it is advantageous to limit the knee bending or flexion, to only slightly dampen or even support the extension movement and, if necessary, to support hip flexion if, on the basis of the detected rotation and the respective position the upper part and lower part in relation to each other, it is detected that the knee joint should be extended or sufficiently extended before the foot is set down. This type of control is also advantageous for certain sports involving frequent changes of direction.

In one embodiment, the at least one sensor is designed as an IMU and is attached to the upper part or the lower part of the treated or untreated contralateral limb or to the torso of the user and is coupled to the control device. With the IMU or with several IMUs, it is possible to obtain information regarding the orientation not only with respect to the rotation about the longitudinal axis, but also about other axes, and with respect to the movements in different planes. In addition, it is possible to record positions, orientations and/or accelerations of the upper part and/or the lower part or to calculate them on the basis of the sensor data. If several IMUs are used, one of which is assigned to the upper part and the other to the lower part, angles between the components can be calculated from the determined absolute angles or spatial position angles in the respective planes.

In a further development, at least one force sensor, acceleration sensor, angle sensor and/or moment sensor is arranged on the upper part and/or the lower part. A force sensor can be designed, for example, to detect contact with the ground. A compressible element, a deformable or displaceable element or even an elastically mounted element can act on a force sensor or on a contact switch serving as a force sensor, in order to detect, for example, whether the respective leg is in a stance phase or in a swing phase.

A change in orientation about the longitudinal axis can be both a rotation of a component about a longitudinal axis and a changing direction of movement of a component during movements along a curved trajectory. A change in orientation about a longitudinal axis can therefore also be a curved trajectory of a component in the transverse plane. A change in orientation can also be a superimposed movement of a rotation and of a curved trajectory. The orientation can be the current direction of movement during movement along a curved trajectory. The orientation about a longitudinal axis is accordingly the direction of movement in the transverse plane. A transverse plane is a plane normal to a longitudinal axis. A rotation or rotational movement about a longitudinal axis is equivalent to such a change in orientation about the longitudinal axis.

During a rotation about the longitudinal axis of the longitudinal extent in the proximal-distal direction of the orthopedic device or of the contralateral side or of the body, the longitudinal axis can refer both to the current longitudinal axis, which is pivoted too when the orthopedic device and/or the contralateral side is pivoted in the sagittal plane and/or frontal plane, and to the longitudinal axis in a reference position, for example when standing upright.

If the orthopedic device is controlled on the basis of an orientation and/or change in orientation about a longitudinal axis, it is possible that a certain minimum amount of movement or change in movement over time, and any derivatives thereof, are necessary in order to have an influence on the control. This can be implemented using one or more threshold values and/or using more complex algorithms, e.g. a majority decision over several values, or using artificial intelligence.

All control algorithms which have an orientation and/or change in orientation about a longitudinal axis or variables derived therefrom as input variables can also have other input variables, in particular movements in other directions, loads and/or information from other sensors which influence the behavior of the orthopedic device.

Exemplary embodiments of the invention are explained in more detail below on the basis of the figures. In the figures:

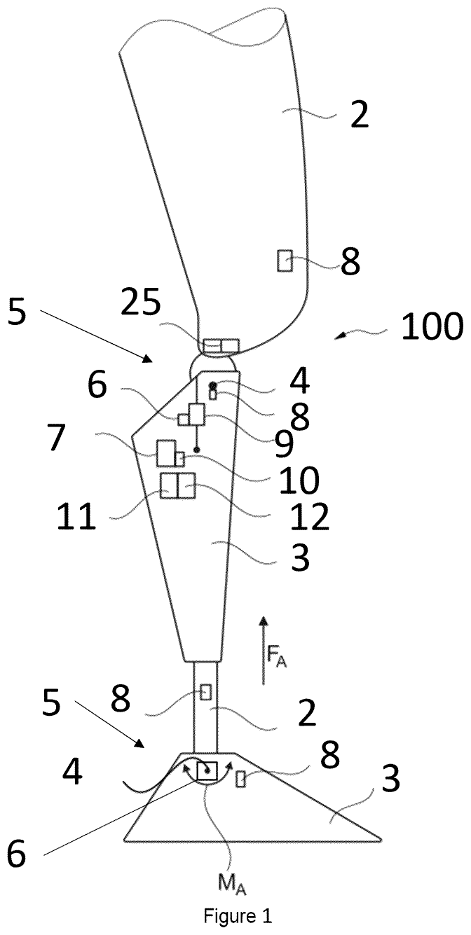

FIG. 1 shows a schematic illustration of a prosthetic leg;

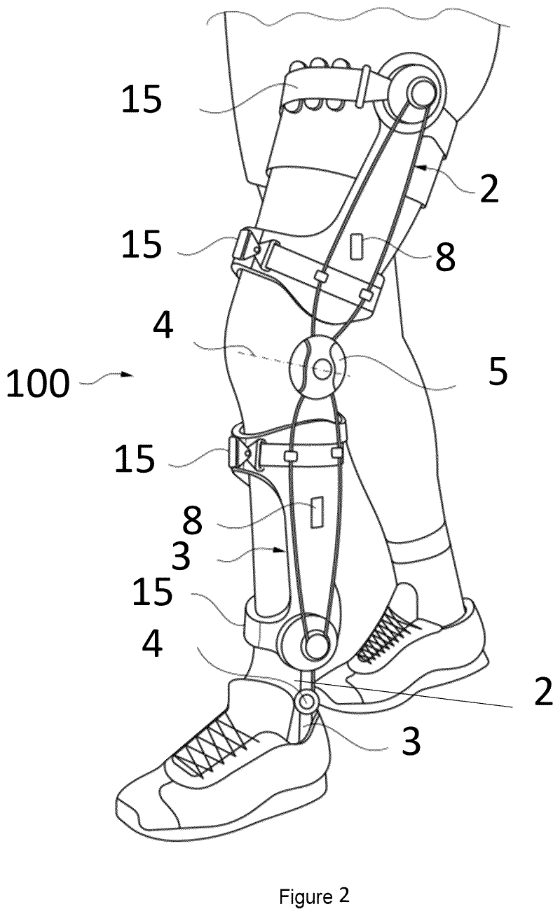

FIG. 2 shows a schematic illustration of a knee-ankle-foot orthosis;

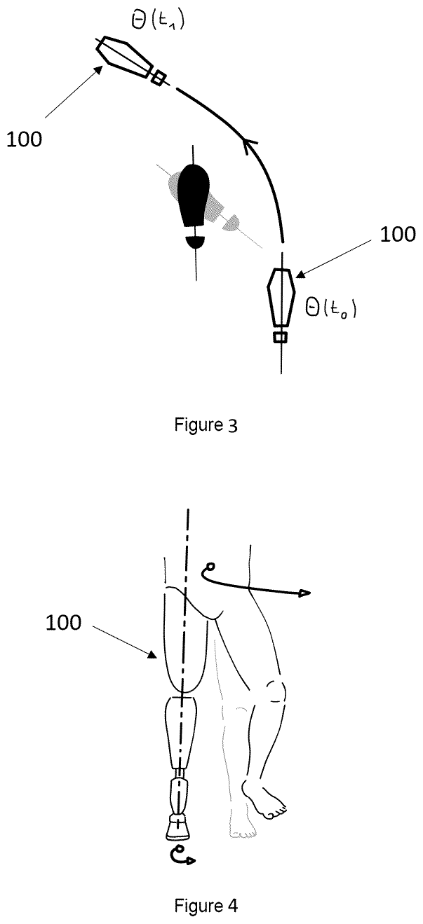

FIG. 3 shows a first movement sequence for walking around a bend;

FIG. 4 shows a schematic frontal view of a second state;

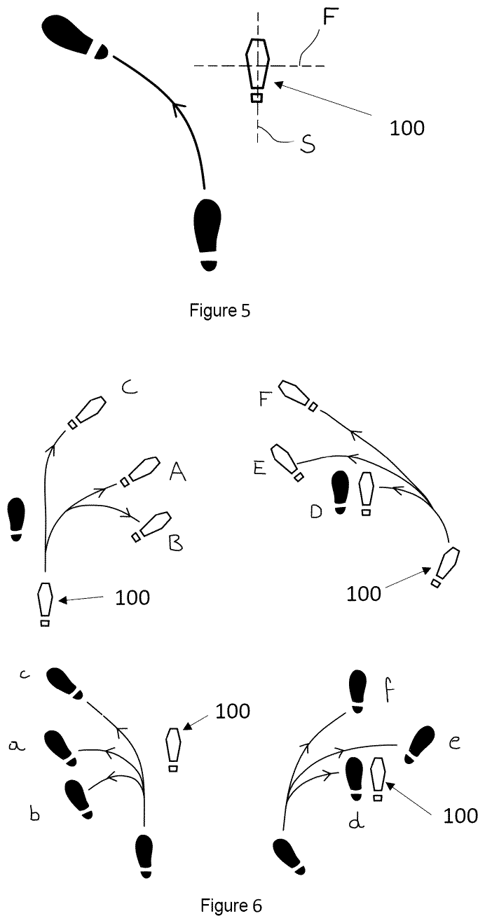

FIG. 5 shows a second movement sequence from the state of FIG. 4;

FIG. 6 shows different movement sequences;

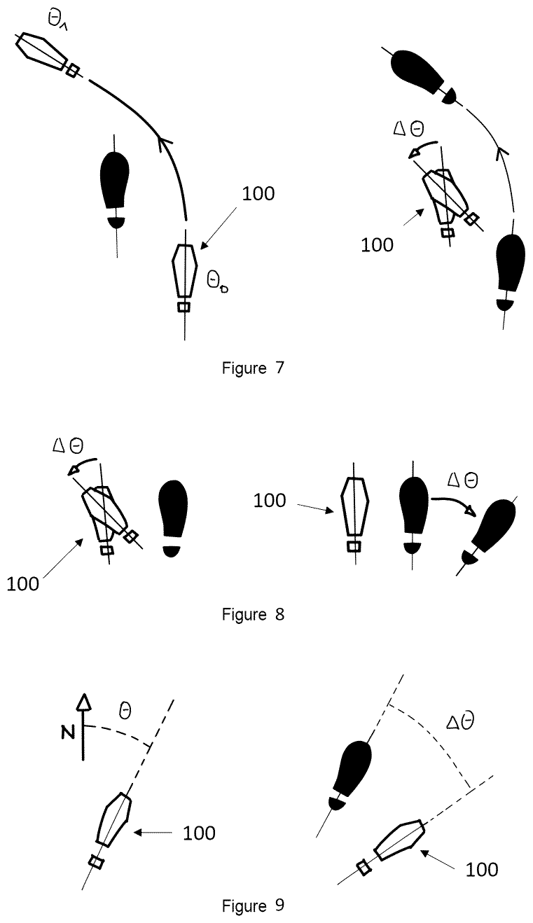

FIGS. 7 and 8 show further embodiments of movement sequences;

FIG. 9 shows a detection of the orientation in the transverse plane;

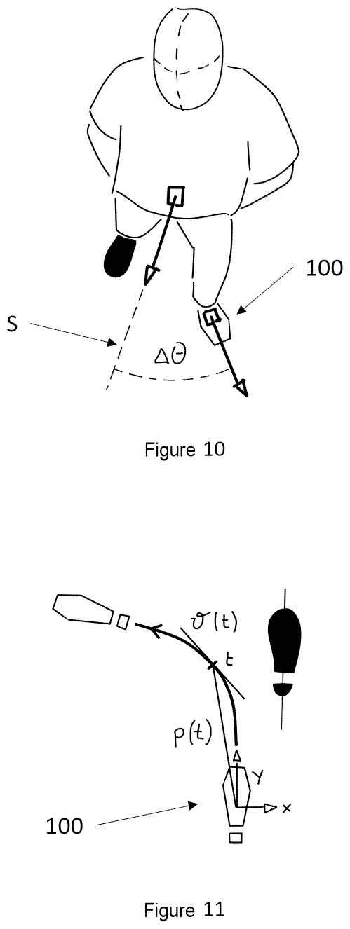

FIG. 10 shows an illustration of a relative rotation of the torso and ipsilateral side;

FIG. 11 shows a trajectory of an ipsilateral side;

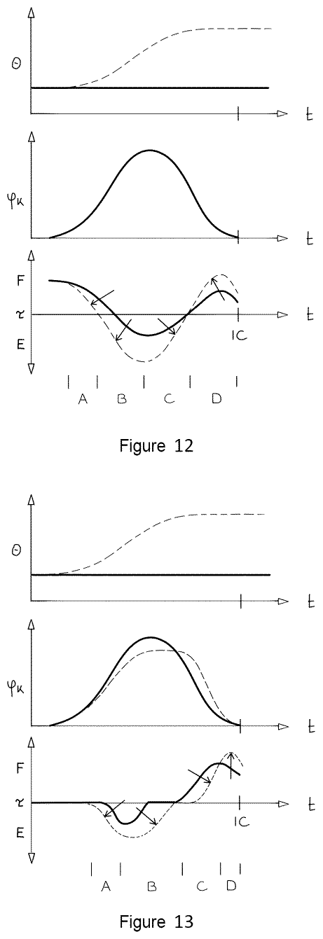

FIGS. 12 to 14 show various parameter sequences;

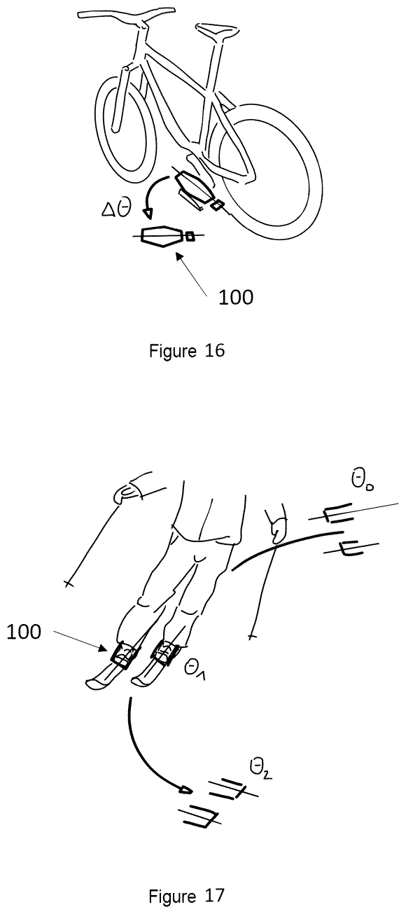

FIG. 15 shows an illustration of a movement sequence during rotation on a staircase;

FIG. 16 shows an illustration of a deactivation during a rotation;

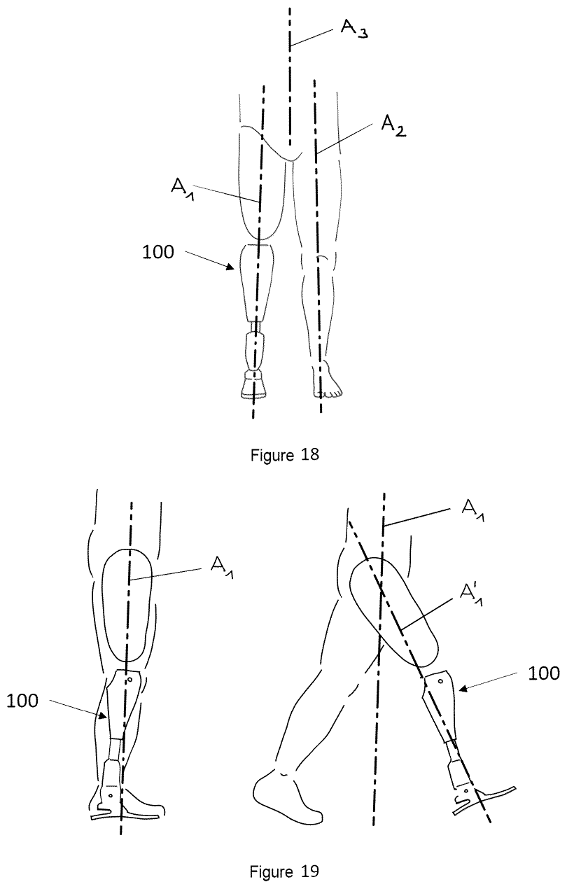

FIG. 17 shows an application example for control adaptation;

FIG. 18 shows an illustration of rotation axes in the frontal plane;

FIG. 19 shows an illustration of rotation axes in the sagittal plane; and

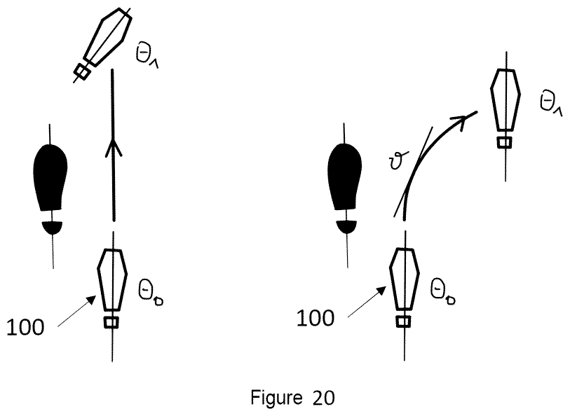

FIG. 20 shows two further movement sequences.

FIG. 1 shows a schematic illustration of an orthopedic device 100 in the form of a prosthetic leg with a first upper part 2 in the form of a thigh socket and a first lower part 3 in the form of a lower part of a prosthetic knee joint 5. The upper part 2 is mounted pivotably relative to the lower part 3 of the prosthesis about a pivot axis 4. On the upper part 2, fastening devices 25 are arranged or formed for securing the thigh socket to the prosthetic knee joint 5. The fastening devices 25 are, for example, a pyramid adapter with a corresponding receptacle. The first lower part 3 in the form of a lower-leg part has a lower-leg tube at its distal end, which in turn serves as second upper part 2 for an articulated connection to a prosthetic foot as second lower part 3. The prosthetic foot 3 is mounted pivotably about the ankle joint axis as second pivot axis 4. The pivotable connection of lower-leg tube and prosthetic foot forms the ankle joint 5. Thus, the orthopedic device 100 has two upper parts 2 and two lower parts 3, wherein the lower-leg part, as the connection between the two pivot axes 4, can be formed in one part or multiple parts and, depending on the way it is considered, is once the lower part and once the upper part.

The articulated connection of upper part 2 and lower part 3 about the respective pivot axis 4 forms the respective joint 5. In the exemplary embodiment shown, a resistance device 9 in the form of an adjustable damper is arranged between the upper part 2 and the lower part 3 of the knee joint. The resistance device 9 is supported on the upper part 2 by a proximal connection device and on the lower part 3 by a distal connection device. In the exemplary embodiment, the resistance device 9 is designed as a passive component and influences a pivoting movement of the upper part 2 relative to the lower part 3 about the pivot axis 4 in both the flexion direction and the extension direction by converting kinetic energy into thermal energy. The resistance device 9 is assigned an actuator 6 for adjusting the respective resistance. The actuator 6 acts on the resistance device 9 according to the operating principle. If the resistance device 9 is designed, for example, as a pneumatic or hydraulic damper device, the actuator 6 changes the flow cross-section of the line from an extension chamber to the flexion chamber and back, in order thereby to increase and decrease the respective flow cross-section of an overflow channel. This reduces or increases the flow resistance. Alternatively or in addition to changing the flow cross-section, the actuator 6 or an actuator 6 can be designed as an adjustable magnet, e.g. as an electromagnet that acts on a magnetorheological fluid. By changing the magnetic field, the viscosity of the magnetorheological fluid changes, so that the resistance to pivoting is changed by changing the viscosity. The resistance device 9 can also be designed as an electric motor that can be operated in generator mode, in which flexion resistance and/or extension resistance is changed by a corresponding generator control. In this case, the generator is usually the actuator. If a purely mechanical brake, for example a friction brake, is provided in which brake pads are pressed against a moving component, the actuator is the motor or drive with which the brake pads are pressed against the component.

Alternatively or in addition to a purely passive design of the resistance device, the actuator 6 can also be designed as an active element, e.g. as an electric motor, in order not only to influence a movement of the upper part 2 relative to the lower part 3, but also to actively cause it. Alternatively to a design as an electric motor, the actuator 6 can also use other drive devices or drive principles to release stored energy.

The actuator 6 is activated, deactivated or modulated via a control device 7. Depending on the signal from the control device, the flexion and/or extension is affected and, if necessary, blocked. The control device 7, with the corresponding signal, sets the movement behavior of the respective joint 5 during walking, standing or other use. The control device 7 is assigned sensors 8, which are arranged on the entire orthopedic device 100. The sensors 8 deliver corresponding data wirelessly or via cable connections to the control device 7. The data of the sensors 8 can be pre-processed and/or processed in the control device 7 itself. Processors, memories and all other necessary components are present in the control device 7 or are coupled to it in order to evaluate the sensor data and, on the basis of this evaluation, to carry out a corresponding activation, deactivation or modeling of the actuator and thus of the resistance device 9.

The control device 7 also has, in particular, a storage device 10 and can be coupled to a transmitter 11 and a receiver 12 in order to transfer sensor data, programs, access rights, settings, changes of settings, updates or other things to external components or to components within the orthopedic device. The sensors 8 detect all relevant parameters during use of the orthopedic device, for example forces, moments, accelerations, temperatures, times, orientations in space, deformations, periods of movement, periods of use, distances, relative movement, interactions with the environment, voltages, currents, biosignals, electromagnetic radiation and the like. In particular, the sensors 8 or sensor devices are designed as components that detect an angular position of the components to one another and/or a spatial position or orientation in space. In addition, the sensors 8 are designed to record axial forces FA and moments MA. The forces and moments are determined wherever recording is useful and necessary, even if these forces and moments are only shown in connection with the ankle joint. Not all sensors 8 can record all parameters; the arrangement and design of the sensors depends on the parameters to be recorded in each case.

Derived variables can also be calculated from sensor values. For example, lever arms at certain points and/or force application points can be calculated from force and/or moment components, sensor values can be fused to form characteristic variables, for example in IMUs (inertial measurement units), forces can be back-calculated from deformations, and/or a position can be calculated back from several distances using triangulation. Such calculated variables are included in the embodiments described and can be used to control the orthopedic device, in particular to control movement sequences with a pivoting in the frontal plane.

In the exemplary embodiment, an electric motor is arranged on the ankle joint as actuator 6, via which, according to requirements, a resistance device is provided via the generator operation and, in motor operation, a support or an active displacement of the prosthetic foot relative to the lower-leg part about the pivot axis 4 is provided.

FIG. 2 shows an orthopedic device 100 as a lower-limb orthosis in a fitted state. This is a knee-ankle-foot orthosis (KAFO), in which a first upper part 2 in the form of a thigh rail is secured to a thigh via fastening devices 15 in the form of straps. A first lower part 3 in the form of a lower-leg rail is also arranged via fastening devices 15 on a lower leg of a user. The thigh rail and the lower-leg rail are fastened pivotably to each other about a pivot axis 4 so as to form an orthotic knee joint 5. The components and technical devices explained in FIG. 1, such as actuator, resistance device, control device, interfaces and the like, are arranged on or in the orthotic knee joint 5. The sensors 8 are shown schematically. The second pivot axis 4 in the region of the natural ankle joint connects the lower-leg rail as second upper part 2 to a foot part as second lower part 3. The device for influencing the prosthetic ankle joint as regards the resistance in the direction of plantar flexion or dorsal flexion is housed in the region of the orthotic ankle joint. Passive resistance devices and/or active drives or actuators can also be provided here.

Both in the design as a prosthesis and in the design as an orthosis, with several joints 5 and corresponding resistance devices, the actuators 6 for influencing the pivoting movement about the respective pivot axis 4 can be controlled by a common control device 7. It is also possible for several control devices 7 to be designed or arranged in order to control the orthopedic device 100 accordingly.

FIG. 3 shows schematically a first movement sequence of a person with an orthopedic device 100 in the form of a prosthetic leg, similar to that in FIG. 1. The person using the orthopedic device 100 is standing essentially straight and upright. In the position shown, the left leg, as seen from the user, is the untreated leg. A forward movement would be a movement straight ahead. If the user now wishes to make a change in movement or to turn left from a standing position, at a time t=0 the right leg, which is treated, is first raised with the orthopedic device 100, moved forward and to the left in an arc, and set down at a time tᵢ.

After setting down the prosthetic foot of the orthopedic device 100, the user shifts their weight to the treated side and pulls their left, untreated foot next to the prosthetic foot. In doing this, or during the pivoting movement of the treated side about the longitudinal axis of the supporting leg, the left foot of the supporting leg can also be pivoted. To do this, the forefoot is usually loaded and a rotation about the longitudinal axis of the longitudinal extent of the supporting leg is carried out on the ball of the foot. This is indicated by the footprints shown one above the other. The rotation about the longitudinal axis of the longitudinal extent of the supporting leg or the contralateral side is recorded, for example, via an IMU that is arranged on the orthopedic device 100. If another IMU is fastened to the contralateral limb, a rotation of the treated side relative to the untreated side can also be recorded, so that it is taken into account that a rotation around the supporting leg is also carried out. If a rotational movement about a longitudinal axis of the longitudinal extent is detected, the flexion resistance can be reduced, for example, in order to enable increased knee flexion. This means that a longer distance has to covered between raising the treated side and setting it back on the ground at the time tᵢ, resulting in a delayed or longer movement sequence. After lifting the prosthetic foot, a flexion movement within the knee is facilitated or initiated, for example, by reducing the flexion resistance or by initiating active flexion support using the actuator (not shown). The lifting is detected, for example, by monitoring the axial force curve within the lower part of the prosthesis or within the prosthetic foot in conjunction with monitoring a movement and/or position of the orthopedic device 100. The speed of rotation about the longitudinal axis of the longitudinal extent of the untreated side can be used to estimate how long the period between lifting (t₀) and setting down (tᵢ) the foot will be, so that an increase or decrease in the flexion resistance and/or a decrease in an extension resistance or an activation of a drive can take place. For this purpose, a corresponding signal is generated via the control device and transmitted to the actuator. In one embodiment of the method, the flexion resistance in the artificial knee joint is increased and/or an extension in the artificial knee joint is effected as soon as an axial load or a setting-down of the foot of the treated side is detected. This eliminates the need to estimate the duration of movement, since it is always recognized when a rotational movement about the supporting leg is completed.

During a movement of the prosthetic foot, one embodiment of the method provides for preventing plantar flexion and also for causing dorsal flexion, so that the prosthetic foot or a foot plate of an orthosis can be set down over its entire surface or with a straight sole essentially parallel to the ground. Alternatively, the movement from position t₀ to position t can be combined with plantar flexion in the case of an active foot, so that a tip of the foot sets down first and dorsal flexion occurs as the load increases. It is also possible for the foot to be held in a slightly downward position during the movement or to be brought into this position.

FIG. 4 shows an alternative position in which the treated side with the orthopedic device 100 represents the right leg. The left leg as the contralateral side is raised, so that the treated side represents the supporting leg. If the untreated, left side is now moved in such a way that the foot is placed and twisted forward and to the left, this means that a rotational movement has to take place about the longitudinal extent of the supporting leg. To facilitate this rotational movement, resistance in an ankle joint or an artificial knee joint is reduced about a longitudinal axis and a corresponding rotation is permitted or facilitated. This makes it easier to rotate the thigh and thus the entire torso when a prosthetic foot is set down. If the rotation about the longitudinal axis is detected or a rotation of the untreated side is detected, in one embodiment the flexion resistance within the artificial knee joint is adjusted, for example increased, in order to increase the stability, so that unwanted bending is prevented. To facilitate the rotational movement, in one embodiment an extension movement is supported or an extension resistance is at least reduced so that the treated side can be moved more easily into maximum extension.

FIG. 5 shows a schematic illustration of the movement pattern of a curved movement with a prosthetic foot set down. The prosthetic foot of the orthopedic device 100 is in a position that points forward in the direction of walking, the longitudinal extent of the prosthetic foot lies within the sagittal plane S, and the frontal plane F runs perpendicular thereto. If the lifting of the left, untreated foot is detected or can be inferred, for example from an increase in the axial load in the orthopedic device 100, the corresponding control signals are sent to the actuator. If at the same time a rotation of the untreated side and/or a rotation about the longitudinal axis of the longitudinal extent of the treated side is detected via the sensors, the mobility about the longitudinal axis in the artificial joints can be increased, for example by the reduction of corresponding resistances. In one embodiment, the flexion resistance about the knee axis against dorsal flexion in the ankle joint is increased, in order to avoid unwanted bending in the respective joints and to make the change of direction easier. In the illustrated external rotation with the contralateral side, it is advantageous if the user is able to push off from the treated side in order to bring about the change in momentum. In addition, a slight forward roll-of movement is necessary on the treated side. This is achieved by increasing the resistances. If, after the untreated side has been set down, the orthopedic device 100 is adjusted and moved again parallel to the forward, left foot, a modification of the extension resistances and flexion resistances or of the active pivoting in the extension direction and flexion direction can be carried out for this movement, which can take longer than a normal step when walking straight ahead and in which other forces and moments can act on the auxiliary device than when walking straight ahead.

FIG. 6 shows different movement patterns for a combination of rotational movements, forward movements and lateral movements within the transverse plane. These are therefore curvilinear movements or curve movements in the transverse plane. In the upper row of movements, the movements are carried out by the treated side and are indicated with upper-case letters; in the lower row, the treated side is the supporting leg and the untreated side is moved, which is indicated with lower-case letters. In principle, the movements can also be reversed or the other way round. In movement A, with a prosthetic foot standing behind, for example from a step position with the untreated side at the front, the treated side is moved both forward and sideways up to the level of the untreated side, resulting in a curved movement. In movement B, the forward movement is reversed so that the treated side, for example the prosthetic foot, is placed diagonally behind the untreated foot. In movement C, the treated side is guided in a straight line in the sagittal plane up to the level of the untreated foot and then set down diagonally forward. In all three movements, the treated side is pivoted about a longitudinal extent of the longitudinal axis of both the treated and the untreated sides.

In movements D to F, the starting position of the treated side with a prosthetic foot is diagonally behind the untreated side. In movement D, the treated side is moved to the side of the untreated foot in a circular motion, and in movement E it is moved in front of the untreated foot as part of a cross step. In movement F, the treated foot is crossed and placed diagonally in front of the untreated foot. In all movements, the treated side in the starting position is located, on account of the wide-legged stance, in a position inclined in the frontal plane and performs a rotational movement or a movement on a curved path that deviates from the usual movement pattern of walking forwards and has to be countered with appropriately adapted control of the resistances and/or drives.

The same or corresponding movements are carried out in the lower row with the untreated side; the prosthetic foot or the foot part of the treated side is the standing component. The movements are carried out accordingly and can be carried out in both directions, i.e. a moving away instead of a pulling toward, and vice versa. During movements with the untreated side in the swing phase, a rotation takes place in the ankle joint with a fixed position, a rotation about a foot contact point or a COP or in the hip joint of the treated side by rotation of the entire of torso.

By taking into account rotational movements about the longitudinal axis of a longitudinal extent of an orthopedic device, a contralateral side or a rotation of several components or limbs relative to each other and, if necessary, the entire torso, it is possible to take into account the special conditions for movements that deviate from walking on a level surface in a straight direction. Especially when walking round bends, the untreated side is often used as a supporting leg, so that an internal rotation about the untreated side is carried out. The rotational movement carried out in the swing phase of the treated side causes different forces and moments to act on the joints of the orthopedic device than when walking straight ahead, which leads to the need to adapt the standard control. The rotational movement can, for example, cause the lower leg to swing up more or the knee joint to remain in the bent position for longer, so that there is a risk that the foot on the treated side will not be in the intended position in time for initial contact. The way in which the pivoting movements of the joints are influenced by resistances and drives must therefore be changed so that the joint is extended in good time before initial contact. For example, the joint can be extended more quickly or the flexion resistance can be increased in order to prevent excessive or prolonged swinging. Using information concerning the orientation or the change in orientation about the longitudinal axis of the longitudinal extent of the orthopedic device, the contralateral side or a reference orientation, it is possible to optimize the control of the resistances and drives. The orthopedic device can include either passive or active joints or joint systems.

It is particularly advantageous to change the resistance or to activate or deactivate drives in the orthopedic device in the swing phase of the treated side when the latter is moved without contact with the ground. In movements that involve a rotation about a longitudinal axis that runs essentially proximal to distal, the orthopedic device located under the body behaves differently than in the case of rectilinear movements, for example in the sagittal plane. A rotation is an accelerated movement that particularly influences the pendulum duration. Whereas in a rectilinear movement the entire extent of the walking movement is limited by the rolling movement of the contralateral side and the step length, rotational movements can take significantly longer, even at high rotational speeds, for example when reversing the walking direction or when rotating once or multiple times around one's own axis. The resistances are adapted or the drives activated and deactivated not only during repeated gait cycles, but also during movements in which the orthopedic device is repositioned under the body without any load. The resistances or drives are also adapted when walking with rotational movements on ramps or stairs.

Ankle joints can also be modified in terms of their resistance or mobility when rotations about a longitudinal axis, for example about the longitudinal axis of the lower-leg part, are detected. If there is a degree of freedom of rotation about the longitudinal axis of the lower leg or lower-leg part, it can be increased if an internal rotation or external rotation is detected. During external rotation of the thigh, either absolutely or relative to the body, the foot can also be rotated outward; vice versa in the case of an internal rotation. This can be advantageous, particularly in the case of an external rotation on the contralateral supporting leg, with the external rotation and internal rotation occurring for the ankle joint both in the swing phase and in the stance phase.

In one embodiment, only the relative change in orientation between two points in time is measured, for example between toe-off and the renewed set-down of the foot. If the rotation between these two positions is more than a threshold value, for example more than 90°, the control is adjusted; an absolute determination of the cardinal direction or orientation with respect to a reference value is not necessary.

FIG. 7 shows, on the left, a schematic illustration of another movement sequence, in which a rotation takes place in the ipsilateral swing phase of the orthopedic device 100 or of the limb on which the orthopedic device 100 is arranged. At a first point in time, there is a first orientation θ₀. Both feet are in an almost parallel orientation toward the sagittal plane in step position. The side treated with the orthopedic device 100 is initially raised in the orientation θ₀ and, starting from the initial orientation, is moved forward and approximately 60° counterclockwise around the treated, contralateral side. The final orientation θ₁ at a second point in time is shown in the upper left of the figure. On the right in FIG. 7 the same movement or a corresponding movement of the untreated right leg is shown. The orthopedic device 100 on the treated leg performs a rotation on the supporting leg by an angle Δθ, while the contralateral leg is in the swing phase. In both cases or in both movement sequences, walking takes place on a curved path or a bend, and an orientation or change in orientation about the longitudinal axis of the longitudinal extent of the orthopedic device or of the contralateral side is also detected. During the movement shown on the left in FIG. 7, the orthopedic device 100 is pivoted about the longitudinal axis of the longitudinal extent of the limb on the contralateral side and thus also moved on a circular path, while in the illustration on the right the orthopedic device 100 is only rotated about the longitudinal axis of the longitudinal extent. The longitudinal axis of the longitudinal extent of the orthopedic device 100 in the illustration on the right thus remains stationary, whereas in the illustration on the left it is non-stationary, since a rotation takes place about the contralateral axis.

FIG. 8 also shows two situations in which a rotation takes place about the longitudinal axis of the longitudinal extent of the orthopedic device 100. In the illustration on the left, a rotation takes place on the ipsilateral side when standing on both legs. Both feet are on the ground; the left, treated side is rotated about the longitudinal axis of the longitudinal extent, in the illustrated embodiment counterclockwise by the angle Δθ. It is also possible that the treated side is relieved of pressure or slightly raised during the rotation in order to make the rotation easier. In principle, it is also possible that a rotation of the contralateral, untreated side is detected from a standing position, as is shown in the illustration on the right, from which conclusions can be drawn concerning possible or future movements on the treated side. Walking in the narrower sense does not take place or does not yet take place; rather a preparation for walking or a re-orientation of the person and of the orthopedic device. If detected early, the control of the orthopedic device can already be adapted to a subsequent movement, for example in order to enable initiation of a subsequent swing phase and to control it accordingly.

It is also possible that when a rotation is detected from a standing position or from the stance phase, no initiation or activation of a swing phase takes place, so as to avoid unexpected or uncontrolled bending in such situations. In the event of rotation, switching to a special mode, for example for climbing stairs, sitting down or standing up, can also be prevented or aborted, in particular so as to prevent accidental switching to a special mode or to switch back in good time to an initial mode. Accordingly, it is advantageous to detect such a rotation and adjust the control.

FIG. 9 shows an example of how the orientation of the orthopedic device 100 can be detected or determined. The orientation in the transverse plane can, for example, refer to an external reference value, for example a magnetic field such as the earth's magnetic field, as is shown in the left-hand illustration of FIG. 9 using the north orientation as an example. The orientation of the longitudinal extent, for example of the foot of the orthopedic device, is then determined in relation to the north-south orientation of the earth's magnetic field. Any suitable reference system that offers sufficient accuracy and stability can be used. Alternatively or additionally, it is possible to determine a rotation about a certain angle Δθ in relation to the contralateral side and to use it for the control. As reference value it is possible to use, for example, the foot, the lower leg or the thigh of the contralateral side, for example an untreated side, or a side that is also treated but is not relevant in terms of the control. The orientation of the treated side relative to the contralateral side can be determined, for example, by sensor devices that record the distance, the changes in distance, and movements of the contralateral side and ipsilateral side relative to each other. Transmitters or markings, for example on the shoe or on the foot part, can be arranged on the contralateral side, the orientation of which transmitters or markings relative to reference points or other detection elements on the ipsilateral side is recorded and evaluated. By recording the rotation of the contralateral and ipsilateral sides, in each case with respect to a common reference, the relative rotation from ipsilateral to contralateral side can be determined. The relative orientation from ipsilateral side to contralateral side can also be determined using at least two IMUs, for example one IMU mounted on the ipsilateral side and one on the contralateral side, and/or one or more relative angle sensors.

FIG. 10 shows a schematic illustration of the situation of a person using an orthopedic device 100 in which a rotation has been effected about the longitudinal extent of the longitudinal axis of the orthopedic device 100, which is oriented, for example, essentially in the direction of gravity. FIG. 10 shows how a relative rotation of the treated side or of the orthopedic device 100 relative to the torso of the user can be determined and used for the control. The sagittal plane S is shown schematically along the forwardly directed orientation. If the treated side or orthopedic device 100 is in an initial position in which, for example, the foot part is oriented forward and runs parallel to the sagittal plane S, the rotation Δθ relative to the torso of the user can be determined and used for the control. The determination takes place, for example, via at least two IMUs or at least one relative angle sensor.

FIG. 11 shows the trajectory of a rotational movement in which the treated side is in the swing phase. A movement pattern, such as that shown for example in the left-hand illustration in FIG. 7, can be determined in terms of the trajectory. The respective trajectory of the ipsilateral side, for example of a foot part, a lower leg or a knee, is determined, for example, via path integration from IMU data and used for control. The trajectory is the sequence of positions p(t) at successive points in time t. In particular, the trajectories in the transverse plane or the projection onto the transverse plane provide information concerning a rotation about a longitudinal axis. The trajectory can be used as an absolute value, or as a relative value in relation to another component or the contralateral side, for the control. FIG. 11 shows the position p of the foot at a time t approximately halfway through the movement from a starting position at the bottom right to a final position at the top left, and also the trajectory in the transverse plane as a sequence of positions p(t). The position is shown relative to the starting position of the ipsilateral foot in a Cartesian, stationary coordinate system. In addition to the position, the current direction of movement and its profile can also be determined. During a rotation, the direction of movement in the transverse plane changes during the course of the movement. In FIG. 11, the current direction of movement ϑ is shown as a tangent to the trajectory at the time t. The direction of movement in the illustration rotates counterclockwise, starting from a movement forward. A rotation or change in the direction of movement can also be understood as a rotation about a longitudinal axis. By recording the changing direction of movement, it is possible to conclude that a rotation has occurred and to adjust the control of the resistances and drives. The direction of movement can be determined relatively, for example in relation to the contralateral side, but also absolutely, in relation to a reference system.

In FIG. 12, three different parameters are plotted over time, namely the orientation θ, the knee angle φₖ, and the knee moment τ applied by the actuator. FIG. 12 shows possible adjustments in the control of an active prosthetic knee joint or active orthotic knee joint in the ipsilateral swing phase, including what is called the pre-swing phase. The orientation θ of the ipsilateral side is straight ahead when walking, as shown by the solid line. The orthopedic device is guided forward in an essentially unchanged manner with regard to the orientation about the longitudinal axis of the longitudinal extent or the leg axis. The orthopedic device remains in an almost constant orientation, θ, for example in the sagittal plane S. The dashed curve shows a rotation starting from the normal position or initial position, in which, for example, a foot part points forward. In the exemplary embodiment shown, the knee angle curve for walking with or without rotation about the longitudinal extent of the longitudinal axis is the same in both situations. The knee moment τ applied by the actuator is changed, which acts once in the flexion direction F or in the extension direction E. Here too, the solid line shows the course when walking straight ahead on level ground, and the dashed line shows the course when a rotation is performed. A bending moment or flexion moment in the flexion direction F is applied by the actuator in the pre-swing phase when walking straight ahead on level ground. Before the maximum knee angle φₖ is reached, the flexion support is first reduced and then reversed, so as to prevent the knee joint from bending too far. An extension moment E is applied to initiate or support a reversal of movement, so that an extension of the knee joint takes place in the swing phase. At the end of the swing phase, a flexion moment F is again applied so as to prevent an unbraked movement into the extension stop. The flexion moment F is either minimized or reduced after full or sufficient extension has been achieved, in order to initiate or enable stance phase flexion. During rotation about the longitudinal extent of the longitudinal axis, a flexion moment can be reduced earlier in the pre-swing phase A, so that the flexion is supported less or for a shorter time, or a flexion resistance or an extension moment is applied earlier (B). The extension moment in the late swing phase flexion B and/or in the swing phase extension C can be applied higher and/or for a longer time, and an increased extension resistance or a flexion moment can be applied at the end of the swing phase D, if this is necessary due to the rotation about the longitudinal extent of the longitudinal axis, in particular to stop a faster extension.

FIG. 13 shows a parameter curve for controlling a passive knee joint in the ipsilateral swing phase including the pre-swing phase. Here too, a solid line represents walking straight ahead, and the dashed line represents rotation of the ipsilateral side. Unlike the case of an active orthopedic joint device, for example a motor-driven artificial knee joint, the knee angle curve in a passive knee joint changes by reducing the maximum flexion and allowing faster extension. The knee moment, in this case a resistance applied by a damper or a braking device, will set in earlier when a rotation is detected in the pre-swing phase or in the swing phase flexion of phase A than when walking straight ahead; higher resistance is also necessary. The resistance will be higher and longer lasting in the late swing phase flexion (phase B), will set in later in the extension phase C and will be lower than when walking straight ahead. For this purpose, a higher resistance against extension in the late swing phase D shortly before the foot is set down is necessary in order to dissipate the energy of the comparatively faster extension.

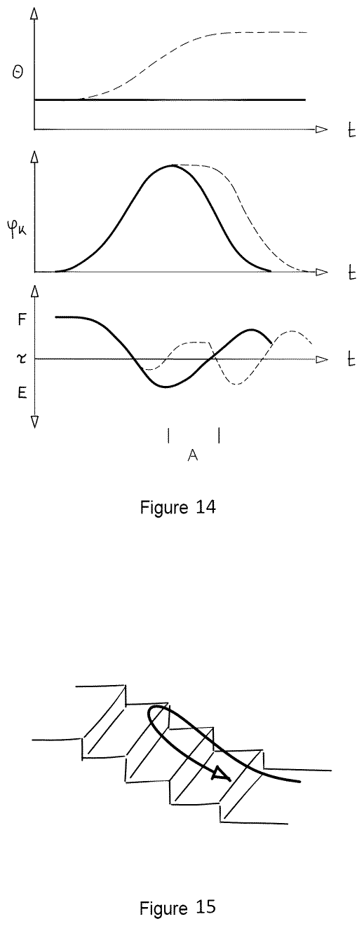

A further variant is shown in FIG. 14, in which the knee angle φₖ is held for longer in the ipsilateral swing phase during a rotation. This can be advantageous, for example, during a slow rotation in which a bent knee joint is held in this position for longer. As an alternative to holding the knee joint in position, a slower extension and/or flexion can also take place. The moment curve of the applied holding moment τ is then changed in such a way that, after the maximum flexion angle or maximum flexion is reached, a flexion moment is generated or an extension moment is reduced in order to keep the knee joint in flexion. In order to initiate an extension or reversal of movement, an extension moment is then applied or an extension resistance is reduced, in order to bring about or facilitate an extension of the knee joint.

FIG. 15 shows a movement sequence in which a special mode for controlling the orthopedic device, for example a prosthetic knee joint or orthotic knee joint, is changed. If it is detected that, in stair-climbing mode, a rotation of the orthopedic device occurs about the longitudinal extent of the longitudinal axis of the orthopedic device and/or a contralateral side, another walking mode can be set. If rotation is detected on a staircase, for example because the foot on the ipsilateral side is not lifted and a rotation takes place, the stair-climbing mode is exited and the system switches for example to a basic mode, the mode for walking on level ground or a mode for walking down stairs. A movement that has already been initiated for placing the foot upward in staircase mode, for example knee flexion, is aborted when rotation is detected, and the leg is extended again to enable the treated side to be placed on the ground and the load to be transferred to it.

FIG. 16 shows a variant in which a special mode, in the example shown for cycling, is deactivated. If getting off a bicycle is detected due to a movement of the treated ipsilateral side, for example via a rapid rotation of the prosthetic foot or of the foot part of an orthosis, the cycling mode is exited. The rotation can, for example, result from the foot being released from a pedal lock. Deactivation of the special mode can be triggered depending on a threshold value being reached. The rotation of the foot has to exceed a certain speed or a relative angle of rotation before the special mode is deactivated, since rotational movements, in particular slow rotational movements, can also occur during a normal movement sequence in the special mode.

A further example of a movement sequence with separate control for an actuator is shown in FIG. 17, which illustrates skiing. If the control detects skiing or if such a special function is activated by the user, the control is adapted depending on the orientation or a change in orientation. For example, with a so-called downhill ski, an extension is initially blocked if it tilts laterally inward and thus toward the slope. This enables particularly good transmission of power. If an outward rotation of the downhill ski is then detected during a change due to a turn, the extension lock is released and extension can take place. A lock can also be activated when changing from uphill ski to downhill ski.

FIG. 18 shows different longitudinal axes in the frontal plane when standing, about which axes a rotation can take place. A1 is the longitudinal axis of the orthopedic device 100. A2 is the longitudinal axis of the contralateral side, which in this case is not provided with an aid. A3 is the proximal-distal axis of the torso.

FIG. 19 shows, on the left, the longitudinal axis A1 of the orthopedic device 100 when standing. If the orthopedic device 100 is pivoted in the course of a movement, which is shown on the right in FIG. 19, the orientation of the longitudinal axis A1 also changes to the longitudinal axis A1′ of the orthopedic device 100 in the pivoted position. In this illustration, a rotational movement points out of or into the plane of the drawing and is therefore not visible. A rotation about a longitudinal axis can now refer both to the current longitudinal extent A1′ of the orthopedic device 100, which rotates in the sagittal plane, and to the longitudinal extent A1 in a reference position, such as in the standing position shown on the left. The same applies to the longitudinal extent of other components and body parts, such as the contralateral leg or the torso.

A rotation can be both a twisting of a component about a longitudinal axis and a curved trajectory in which the direction of movement rotates with the movement. In previous figures, these movements were mostly shown in a superimposed form. However, these two forms of rotation can also occur in isolation. FIG. 20 shows, on the left, a movement in which the foot of the orthopedic device 100 is placed forward in a straight line from a step position and rotates outward (θ₁) clockwise from a parallel position (θ₂). The rotation takes place about the longitudinal axis of the ipsilateral side. In the illustration on the right, the foot of the orthopedic device 100 is placed diagonally outward starting from a step position in which the foot of the treated side is behind the contralateral foot. The foot of the treated side describes a circular path. The orientations of the foot in the starting position θ₀ and in the end position θ₁ are identical. The change in the direction of movement ϑ due to the circular or curved movement is, within the meaning of the invention, a rotation about the contralateral supporting leg. In many cases, a combination of a twisting movement and a curved trajectory occurs, as shown for example in FIG. 6.

1. A method for controlling a lower-limb orthopedic device having a proximal upper part and a distal lower part, which are mounted on each other in an articulated manner about at least one pivot axis so as to form a joint with each other, means for fastening the orthopedic device to a limb, and at least one actuator coupled to a control device which activates or deactivates the actuator on the basis of sensor data from at least one sensor coupled to the control device, in order to influence a pivoting resistance and/or a movement of the upper part relative to the lower part or of two components of the orthopedic device relative to each other, characterized in that an orientation and/or change in orientation about the longitudinal axis of the longitudinal extent in the proximal-distal direction of the orthopedic device and/or of a contralateral limb are detected using the sensor data, and the actuator is activated or deactivated, or a setpoint value for the actuator is modulated, on the basis of the orientation and/or change in orientation about the longitudinal axis.

2. The method as claimed in claim 1, characterized in that the sensor data are determined and the actuator is activated or deactivated, or the setpoint value for the actuator is modulated, during the use of the orthopedic device in the fitted state.

3. The method as claimed in claim 1, characterized in that the orientation or displacement is detected and determined via a spatial position sensor, an IMU and/or angle sensors.

4. The method as claimed in claim 1, characterized in that forces, moments and/or accelerations are detected via sensors and used as the basis for the control.

5. The method as claimed in claim 1, characterized in that the orthopedic device is designed as a prosthesis or orthosis and has an artificial knee joint and/or an artificial ankle joint to which the actuator is assigned.

6. The method as claimed in claim 1, characterized in that the at least one sensor is arranged on the orthopedic device, the contralateral limb or the torso of the user, or in that several sensors are arranged on the orthopedic device, the contralateral limb or the torso of the user and the sensor data are used as the basis for the control.

7. The method as claimed in claim 1, characterized in that, during a rotation of the treated side about the longitudinal axis of the longitudinal extent of the contralateral limb in the swing phase of the orthopedic device, an increase in flexion resistance is initiated and/or a reduction in extension resistance or an extension is initiated.

8. The method as claimed in claim 1, characterized in that hip flexion is supported during a rotation of the treated side about the longitudinal axis of the longitudinal extent of the contralateral limb in the swing phase of the orthopedic device.

9. The method as claimed in claim 1, characterized in that, during a rotation of the treated side about the longitudinal axis of the longitudinal extent of the treated side in the stance phase, the flexion resistance is increased, bending is reduced and/or bending is prevented.

10. The method as claimed in claim 1, characterized in that the orientation and/or change in orientation about the longitudinal axis of the longitudinal extent in the proximal-distal direction of the orthopedic device and/or of a contralateral limb relative to the torso of the patient or to an external reference orientation are detected.

11. The method as claimed in claim 1, characterized in that the actuator is activated or deactivated, or a setpoint value for the actuator is modulated, depending on the duration, extent, speed and/or speed profile of the change in orientation and/or of a movement.

12. The method as claimed in claim 1, characterized in that an IMU is attached as sensor to the treated limb, the contralateral limb and/or the torso of the patient, and a rotation of the limb or limbs relative to the torso is detected and used as the basis for the activation or deactivation or modulation of a setpoint value of the actuator.

13. The method as claimed in claim 1, characterized in that the trajectory in the transverse plane is detected and used as the basis for the activation or deactivation or modulation of a setpoint value of the actuator.

14. The method as claimed in claim 1, characterized in that, during a rotation with the treated side about the longitudinal axis of the longitudinal extent of the treated side in the stance phase (as compared to walking straight ahead), the flexion resistance in the knee joint is not reduced or is reduced to a small extent (less than when walking straight ahead) and/or no flexion movement or a small flexion movement (less than when walking straight ahead) is initiated.

15. The method as claimed in claim 1, characterized in that a special mode is exited and/or not activated during a rotation of the treated side about the longitudinal axis of the longitudinal extent of the treated side.

16. A lower-limb orthopedic device having an upper part and a lower part, which are mounted on each other in an articulated manner about at least one pivot axis so as to form a joint with each other, and at least one actuator coupled to a control device which activates or deactivates the actuator on the basis of sensor data from at least one sensor coupled to the control device, in order to influence a pivoting resistance or a movement of the upper part relative to the lower part, characterized in that the at least one sensor is designed and configured to detect sensor data concerning an orientation and/or change in orientation of the orthopedic device about the longitudinal axis of the longitudinal extent in the proximal-distal direction of the orthopedic device and/or of a contralateral limb, and in that the control device is configured to activate or deactivate the actuator, or to modify a setpoint value for the actuator, on the basis of the orientation and/or change in orientation about the longitudinal axis.

17. The orthopedic device as claimed in claim 16, characterized in that at least one sensor is designed as an IMU and is fastened to the upper part or the lower part, the treated or untreated contralateral limb or the torso of the patient and is coupled to the control device.

18. The orthopedic device as claimed in claim 16, characterized in that at least one force sensor, acceleration sensor, angle sensor and/or moment sensor is arranged on the upper part and/or the lower part.
